SEQUENCE LISTING (8 sequences)

SEQ ID NO: 1 - DNA, 18 bp, Artificial Sequence
  CB1R forward
cgtaaagacagccccaat

SEQ ID NO: 2 - DNA, 18 bp, Artificial Sequence
  CB1R reverse
ctgggtcccacgctgaat

SEQ ID NO: 3 - DNA, 18 bp, Artificial Sequence
  CB2R forward
tttcacggtgtggactcc

SEQ ID NO: 4 - DNA, 18 bp, Artificial Sequence
  CB2R reverse
taggtaggagatcaacgc

SEQ ID NO: 5 - DNA, 20 bp, Artificial Sequence
  Collagen I forward
ggcggccagggctccgaccc

SEQ ID NO: 6 - DNA, 20 bp, Artificial Sequence
  Collagen I reverse
aattcctggtctggggcacc

SEQ ID NO: 7 - DNA, 22 bp, Artificial Sequence
  GAPDH forward
caactacatggtttacatgttc

SEQ ID NO: 8 - DNA, 18 bp, Artificial Sequence
  GAPDH reverse
gccagtggactccacgac

(19) United States
(12) Patent Application Publication
(10) Pub. No.: US 2014/0142106 A1
(43) Pub. Date: May 22, 2014

(54) METHOD FOR TREATING PERITONEAL FIBROSIS

(71) Applicant: TAIPEI VETERANS GENERAL HOSPITAL, Taipei City (TW)

(72) Inventors: Chih-Yu Yang, Taipei City (TW); An-Hang Yang, Taipei City (TW)

(73) Assignee: TAIPEI VETERANS GENERAL HOSPITAL, Taipei City (TW)

(21) Appl. No.: 13/739,825

(22) Filed: Jan. 11, 2013

Related U.S. Application Data
(60) Provisional application No. 61/729,170, filed on Nov. 21, 2012.

(51) Int. Cl. A61K 31/5377; A61K 31/454

(52) U.S. Cl. CPC A61K 31/5377; A61K 31/454
USPC 514/236.5; 514/323

(57) ABSTRACT

The present invention provides a method for treating, preventing or reducing peritoneal fibrosis comprising administering to a subject in need thereof a pharmacologically effective dose of a type I cannabinoid receptor (CB₁R) antagonist or a type II cannabinoid receptor (CB₂R) agonist. Also provided is a dialysis fluid for treating, preventing or reducing peritoneal fibrosis comprising electrolytes, an osmotic agent, a physiologically acceptable pH solution, and a pharmacologically effective dose of a CB₁R antagonist or a CB₂R agonist.

### FIELD OF THE INVENTION

The present invention generally relates to a new method for treating peritoneal fibrosis.

### BACKGROUND OF THE INVENTION

Life-long peritoneal dialysis (PD) as a renal replacement therapy is limited by peritoneal fibrosis. Several studies have shown that hypertonic glucose solution not only is toxic to mesothelial cells [1;2], but also promotes immune cell apoptosis [3]. In addition, the high-temperature sterilization process produces glucose degradation products (GDPs) such as methylglyoxal (MGO), acetaldehyde, formaldehyde, and 3-deoxyglucosone in PD dialysate [4-7]. GDPs possess strong oxidative properties and toxicity, and can induce advanced glycosylated end products (AGEs) [8]. Furthermore, it has been demonstrated that MGO, a key GDP, in PD dialysates inhibits the insulin signaling pathway, resulting in increased endogenous reactive oxygen species production and subsequent cell injury [9]. It has been reported that 2-33 μM of MGO is present in commercial glucose-based peritoneal dialysis fluids [10;11]. After long-term exposure to various GDPs and AGEs, mesothelial cells undergo a dedifferentiation process and peritoneal fibrosis ensues [12-16]. Furthermore, these chronic inflammatory sites are associated with progressive peritoneal angiogenesis [16-19], and finally, PD efficiency is reduced. However, therapeutic strategies targeting these pathogenic processes have not been fully developed [17].

### SUMMARY OF THE INVENTION

This invention is based on the unexpected findings that either intra-peritoneal CB₁R antagonist or CB₂R agonist is effective in ameliorating peritoneal fibrosis in the methylglyoxal (MGO) model.

Accordingly, in one aspect, the present invention provides a method for treating, preventing or reducing peritoneal fibrosis comprising administering to a subject in need thereof a pharmacologically effective dose of a type I cannabinoid receptor (CB₁R) antagonist or a type II cannabinoid receptor (CB₂R) agonist.

In another aspect, this invention also provides a method for treating, preventing or reducing peritoneal fibrosis in a subject under peritoneal dialysis (PD) treatment, comprising: (a) supplementing a peritoneal dialysis fluid (PDF) with a pharmacologically effective dose of a CB₁R antagonist or a CB₂R agonist; and (b) applying such PDF in said PD treatment.

In third aspect of the invention, also presented is a dialysis fluid for treating, preventing or reducing peritoneal fibrosis comprising electrolytes, an osmotic agent, a physiologically acceptable pH solution, and a pharmacologically effective dose of a CB₁R antagonist or a CB₂R agonist.

These and other aspects will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The foregoing summary, as well as the following detailed description of the invention, will be better understood when read in conjunction with the appended drawing. In the drawings:

FIG. 1 shows MGO-induced peritoneal fibrosis in rats. Masson's trichrome stain of rat peritoneal tissues (a-d) [Scale bar: 100 μm]. Morphometric analyses show significant increase of peritoneal thickness in a dose-dependent manner (e) and prominent loss of surface mesothelial cells at a higher concentration of MGO (6-12 mM) (f). Data represent means±SEM (n=6 in each group). Abbreviation: PDF, peritoneal dialysis fluid.

FIG. 2 shows that CBR ligands ameliorate MGO-induced peritoneal fibrosis in rats.

Masson's trichrome stain and morphometric analyses of rat peritoneal tissues show that MGO-induced peritoneal thickening is significantly attenuated either by CB₁R (AM281) treatment (a-d) or by CB₂R (AM1241) treatment (e-h) [Scale bar: 100 μm]. Data represent means±SEM (n=6 in each group). Abbreviation: PDF, peritoneal dialysis fluid.

FIG. 3 shows that MGO-induced mesothelial cell detachment (a) and angiogenesis (b) are attenuated by the selective CB₁R antagonist (AM281) treatment. Data represent means±SEM (n=6 in each group). (c) The representative PCR result of the rat peritoneal tissue shows that the up-regulated TGF-β1, VEGF, and Snail after MGO treatment are attenuated by AM281. Abbreviations: PDF, peritoneal dialysis fluid; VEGF, vascular endothelial growth factor.

FIG. 4 provides CBR expression in mesothelial cell culture. (a) The representative immunofluorescent staining of CB₁R (green FITC) and CB₂R (green FITC) in rat peritoneal tissue, MeT-5A cells, and human peritoneal mesothelial cells (HPMC). (b) The PCR assessment of CB₁R and CB₂R expression after TGF-β1 treatment in MeT-5A cells.

FIG. 5 provides the effects of selective CB₁R antagonist (AM281) on cultured mesothelial cells. (a) AM281 reduces TGF-β1-induced cell elongation in MeT-5A and rat peritoneal mesothelial cell (RPMC) culture. (b) Quantitative immunofluorescent staining analyses of MeT-5A cells show preservation of epithelial markers (E-cadherin and cytokeratin 8/18, green FITC) and suppression of type I collagen (red rhodamine) synthesis in the presence of AM281. (c) Western blot analysis of MeT-5A cells reveals that AM281 silences TGB-β1 activated PI3K.

FIG. 6 provides the effects of selective CB₂R agonist (AM1241) on cultured mesothelial cells. PCR analyses of human peritoneal mesothelial cells (HPMC) show that AM1241 suppresses the type I collagen synthesis at lower concentrations of TGF-β1. Data represent means±SEM.

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

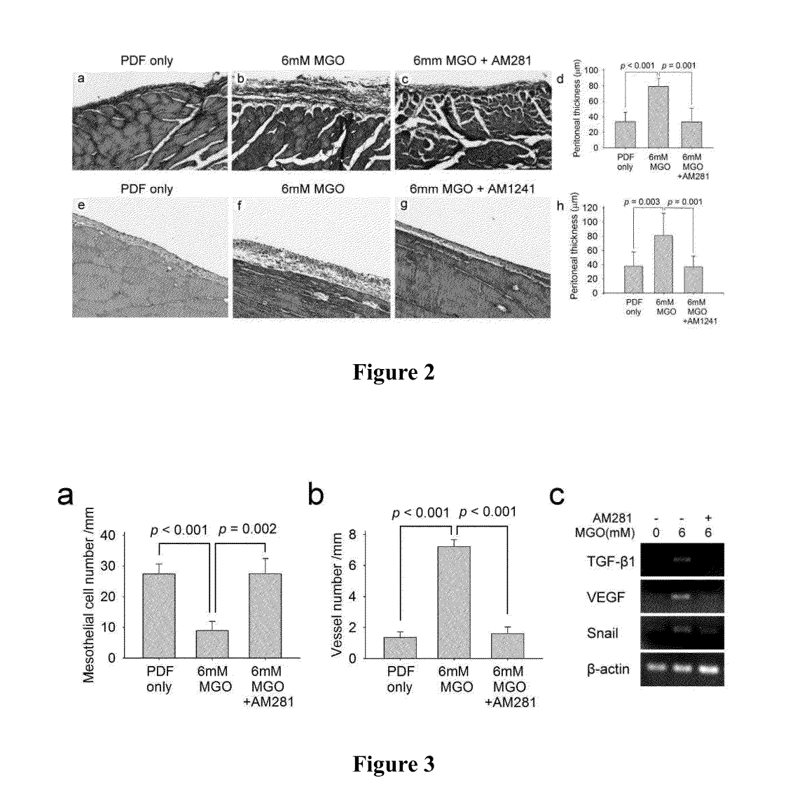

This invention unexpectedly found that CBR ligands have significant effects in reducing peritoneal fibrosis. In the invention particular, it was shown in embodiments of the invention that MGO-induced peritoneal thickening and prominent loss of surface mesothelial cells were attenuated either by the treatment of CB₁R antagonist or CB₂R agonist (FIG. 2). This discovery is especially beneficial to patients suffering from renal failure which requires a life-long peritoneal dialysis (PD) treatment, as long-term PD therapies expose these patients to unwanted risk of peritoneal fibrosis that can result in a shorter lifespan of the peritoneal membrane. Thus, this invention not only offers a potential therapeutic strategy to reduce dialysis-induced peritoneal fibrosis but also prolong the survival in PD patients.

Accordingly, the present invention provides a method for treating, preventing or reducing peritoneal fibrosis comprising administering to a subject in need thereof a pharmacologically effective dose of a type I cannabinoid receptor (CB₁R) antagonist or a type II cannabinoid receptor (CB₂R) agonist.

The type 1 cannabinoid receptors (CB₁R) exist in the brain, and regulate inhibitory neurotransmitters on neurons through psychoactive drug cannabis or endocannabinoids, such as anandamide. Nevertheless, it is recently found that CB₁Rs also exist in tissues other than the central nervous system, and its function varies in different organs [4]. The type 2 cannabinoid receptor (CB₂R) are located on immune cells and modulate cytokine release [20;21]. The CBR ligands, such as cannabidiol, have been proven to be well-tolerated without adverse effects when administered to humans on a long-term basis.

The term “type I cannabinoid receptor (CB₁R) antagonist” as used herein refers to any substances or molecules that block type I cannabinoid receptor selectively. In the invention, the CB₁R antagonist is selected from the group consisting of 5-(4-Chlorophenyl)-1-(2,4-dichloro-phenyl)-4-methyl-N-(piperidin-1-yl)-1H-pyrazole-3-carboxamide (SR141716A), 4-[6-methoxy-2-(4-methoxyphenyl)1-benzofuran-3-carbonyl]benzonitrile (LY320135), N-(piperidin-1-yl)-1-(2,4-dichlorophenyl)-5-(4-iodophenyl)-4-methyl-1H-pyrazole-3-carboxamide (AM251), N-(morpholin-1-yl)-1-(2,4-dichlorophenyl)-5-(4-iodophenyl)-4-methyl-1H-pyrazole-3-carboxamide (AM281), Δ9-tetrahydrocannabivarin (THCV), and analogues thereof. In one specific example of the invention, the CB₁R antagonist is AM281.

The term “type II cannabinoid receptor (CB₂R) agonist” as used herein refers to any substances or molecules that activate type II cannabinoid receptor selectively. According to the invention, the CB₂R agonist is selected form the group consisting of (2-iodo-5-nitrophenyl)-[1-[(1-methylpiperidin-2-yl)methyl]indol-3-yl]methanone (AM1241), (6aR,10aR)-3-(1,1-Dimethylbutyl)-6a,7,10,10a-tetrahydro-6,6,9-trimethyl-6H-dibenzo[b,d]pyran (JWH-133), 1-(2,3-Dichlorobenzoyl)-5-methoxy-2-methyl-3-[2-(4-morpholinyl)ethyl]-1H-indole (GW-405,833), [(1R,2R,5R)-2-[2,6-dimethoxy-4-(2-methyloctan-2-yl)phenyl]-7,7-dimethyl-4-bicyclo[3.1.1]hept-3-enyl]methanol (HU-308), Δ9-tetrahydrocannabivarin (THCV), cannabidiol (CBD), and analogues thereof. In one specific example of the invention, the CB₁R antagonist is AM 1241.

In another aspect, this invention also provides a method for treating, preventing or reducing peritoneal fibrosis in a subject under peritoneal dialysis (PD) treatment, comprising: (a) supplementing a peritoneal dialysis fluid (PDF) with a pharmacologically effective dose of CB₁R antagonist or CB₂R agonist; and (b) applying such PDF in said PD treatment.

Further provided is a dialysis fluid for treating, preventing or reducing fibrosis comprising electrolytes, an osmotic agent, a physiologically acceptable pH solution, and a pharmacologically effective dose of a CB₁R antagonist or a CB₂R agonist.

In one embodiment of the invention, the electrolytes comprise sodium ions, calcium ions, magnesium ions, and chloride ions. One example of the electrolytes as used in the invention comprises about 130-150 mM of sodium ions, 1-2 mM of calcium ions, 0-1 mM of magnesium ions, and 90-110 mM of chloride ions.

The term “osmotic agent” as used herein refers to an agent for maintaining an osmotic pressure as required in a solution. In the invention, the osmotic agent may be selected from the group consisting of monosaccharide, disaccharide, polysaccharide, and amino acid. As an example, the agent is glucose or glucose-derived polymer, such as icodextrin, a type of dextrin which is starch-derived, branched, and water-soluble.

In the invention, the physiologically acceptable pH solution may be any solution having a pH of 4.5 to 7.5. It should be understood by those skilled in the art that the solution can be attained by the use of suitable buffering agents, such as bicarbonates or lactates. In one specific example, the solution has a pH of about 5.

As used herein, the term “pharmacologically effective dose effective amount” refers to an amount effective to treat, prevent, or treat peritoneal fibrosis, which is depending on the mode of administration and the condition to be treated, including age, body weight, symptom, therapeutic effect, administration route and treatment time.

The present invention is further illustrated by the following examples, which are provided for the purpose of demonstration rather than limitation.

### EXAMPLES

### I. Materials and Methods

1. The Animal Model and Tissue Preparation

All animal experiments were performed following the guidelines of the Institutional Committee for Animal Experimentation of Taipei Veterans General Hospital. Five week-old male Sprague-Dawley (SD) rats were purchased from the Laboratory Animal Center of National Yang-Ming University. Peritoneal dialysis fluid (PDF) was prepared according to Hirahara et al. [10;22], containing 2.5% glucose, 100 mmol/L NaCl, 35 mmol/L sodium lactate, 2 mmol/L CaCl₂, and 0.7 mmol/L MgCl₂, in a pH of 5.0. To test MGO effects, fifty mL/kg of PDF containing 0, 3, 6, 12 mM MGO (Sigma, St. Louis, Mo., USA) were injected intra-peritoneally for ten consecutive days. Dimethyl sulfoxide (DMSO) (Sigma, St. Louis, Mo., USA) was used as the solvent of MGO. For investigation of the effects of CBR ligands, SD rats were pretreated with PDF containing either a selective CB₁R antagonist, AM281 (Alexis Biochemicals, Enzo Life Sciences, Farmingdale, N.Y., USA) (1 mg/kg/day) or a selective CB₂R agonist, AM1241 (Sigma, St. Louis, Mo., USA) (1 mg/kg/day) for 3 days followed by continuous treatment of PDF containing respective CBR ligands (AM281 or AM1241) and 6 mM MGO for 10 days. Animals receiving PDF containing DMSO vehicle (without MGO) were used as control (n=6/group). All animals were euthanized at the end of experiments by exsanguinations under general anesthesia.

Certain sections of rat peritoneal tissues were frozen at −80° C. for immunofluorescent analysis, while the remainder were fixed in a neutral formalin solution and then embedded in paraffin for histological analysis. For immunofluorescent staining of CBR, rat brain and spleen were used as the positive control of CB₁R and CB₂R, respectively. For peritoneal thickness quantification, parietal peritoneum sized 1.0×0.5 cm were sampled from four separate peritoneal sites of each rat, including right ventral, left ventral, right lateral, and left lateral abdomen. For each sampling site, the peritoneal thickness was averaged from 15 evenly measured points. Tissue sections were stained with Masson's trichrome to quantify peritoneal fibrosis. The number of mesothelial cells was determined by morphometric method. The hematoxylin and eosin-stained sections were digitalized using an Aperio ScanScope slide scanner (Aperio, San Diego, Calif., USA) at its highest resolution. The mean mesothelial cells per mm surface of peritoneal membrane were counted from 15 points in each peritoneal tissue section from four separate peritoneal areas per animal. The peritoneal angiogenesis was quantified as described by Patel et al. [23]. We performed immunohistochemical staining on 6 μm formalin-fixed and paraffin-embedded rat peritoneal tissue sections following routine procedures. After incubating with primary antibody against α-smooth muscle actin (Abcam, Cambridge, Mass., USA), all sections were labeled using a polymer-HRP staining kit (EnVision, Dako, Glostrup, Denmark). The stained sections were digitalized using an Aperio ScanScope slide scanner at its highest resolution. The mean vessels per mm peritoneal membrane were counted from 15 points in each peritoneal tissue section from four separate peritoneal areas per animal.

2. Cell Culture

The MeT-5A human mesothelial cell line, obtained from the American Type Culture Collection (ATCC, Manassas, Va., USA), was cultured in DMEM (Biological Industries, Israel) containing 15% fetal bovine serum (Thermo Scientific, Waltham, Mass., USA), and 1% penicillin/streptomycin/L-glutamine (Biological Industries, Israel) [24]. The primary culture of human peritoneal mesothelial cells (HPMC) were prepared as previously described [25]. All patient materials were processed following the ethics regulations of the Institutional Review Board of Taipei Veterans General Hospital.

The primary culture of rat peritoneal mesothelial cells (RPMC) were isolated from 5-week-old male SD rats. Trypsin-EDTA (Gibco, Grand Island, N.Y., USA) (1.25%; 100 mL/kg) was injected intra-peritoneally and was then aspirated after a 2-hour dwelling. The aspirate was centrifuged for 10 minutes at 1500 rpm, and was washed with phosphate buffered saline, and then cultured in DMEM containing 10% fetal bovine serum, and 1% penicillin/streptomycin/L-glutamine.

To assess the effects of CB₁R antagonist on mesothelial cells, we pretreated the MeT-5A cells and RPMC with 5 μM of AM281 for 1 hour, and followed by simultaneous AM281 and TGF-β1 (R&D systems, Minneapolis, Minn., USA) treatment for 5 days. Various TGF-β1 concentrations (0.1 to 1.0 ng/mL) were used. In the CB₂R experiment, AM1241 was used instead to test MeT-5A cells and HPMC.

For immunofluorescent studies, we grew mesothelial cells in slide flasks (Lab-Tek II; Nalge Nunc. Naperville, Ill., USA). The primary antibodies and working conditions are listed in the Table 1.

TABLE 1
Primary antibodies and working conditions used for
immunohistochemical analysis

| Source | Working Condition |
|---|---|
| Cayman | 1:100 |
| Santa Cruz | 1:100 |
| Abcam | 1:100 |
| BD Transduction Laboratories | 1:100 |
| Abcam | 1:100 |
| Abcam | 1:100 |

*Abbreviations: CBR, cannabinoid receptor; SMA, smooth muscle actin.

Cells were observed using a confocal LASER microscope (Leica, Wetzlar, Germany), and fluorescent intensity was analyzed using an image-processing software (AlphaEaseFC 4.0, Alpha Innotech, Santa Clara, Calif., USA). The cytoplasmic fluorescent intensity of individual cell is defined as integrated density value (IDV) of selected cytoplasmic area; the average fluorescent intensity of an image field is defined as average density value (ADV=total IDV/total cytoplasmic area).

3. Polymerase Chain Reaction and Western Blot Analyses

For mRNA expression analysis of peritoneal tissue TGF-β1, VEGF, and Snail, the rat peritoneum was resected and incubated with trypsin at 37° C. for 30 minutes. The mesothelium was then scratched from peritoneal surface and collected for reverse transcription-polymerase chain reaction (RT-PCR). In the mesothelial cell culture, the mRNA expression of CB₁R, CB₂R, and type I collagen were also assessed by the PCR analysis. Total RNA was extracted with TRIzol (Invitrogen, Carlsbad, Calif., USA). Contaminated genomic DNA was removed with RNAse-free DNAse (Ambion, Austin, Tex., USA) following the manufacturer's protocol. First-strand cDNA was synthesized from total RNA with Superscript™ first-strand cDNA synthesis system for RT-PCR (Invitrogen, Carlsbad, Calif., USA) using Oligo(dT) as primers. GAPDH was used as control. Amplification was carried out in a thermal cycler (BIO-RAD, MJ Mini, Hercules, Calif., USA) by 30-s denaturation at 94° C., 30-s annealing at 66° C. and 40-s extension at 72° C. Primer sequences are listed in Table 2.

TABLE 2
Primer sequences for reverse transcription PCR

| Target gene | Forward (5′-3′) | Reverse (5′-3′) |
|---|---|---|
| CB₁R | CGTAAAGACAGCCCCAAT | CTGGGTCCCACGCTGAAT |
| CB₂R | TTTCACGGTGTGGACTCC | TAGGTAGGAGATCAACGC |
| Collagen I | GGCGGCCAGGGCTCCGACCC | AATTCCTGGTCTGGGGCACC |
| GAPDH | CAACTACATGGTTTACATGTTC | GCCAGTGGACTCCACGAC |

*Abbreviation: CBR, cannabinoid receptor.

Phosphatidyl inositol 3 kinase (PI3K) activity in the MeT-5A cells was assessed by Western blot. Cell lysates were subjected to SDS-PAGE and probed with primary antibodies against the p110 catalytic subunit of PI3K (Upstate Millipore, Billerica, Mass., USA) or β-actin (Amersham Life Science, Buckinghamshire, UK). The specific labeling of protein was demonstrated by chemiluminescence (ECL-kit, Perkin Elmer, Boston, Mass., USA) and recorded on high-speed film (Kodak Biomax Light Film, Rochester, N.Y., USA).

4. Statistical Analysis

Values of the continuous variables are presented as mean and standard error (SEM), unless otherwise specified. Continuous variables were compared by one-way analysis of variance (ANOVA). SPSS version 15.0 for Windows (SPSS Inc., Chicago, Ill., USA) was used for all statistical analyses. All probabilities were two-tailed and a p value of less than 0.05 was considered to be statistically significant.

### II. Results

1. The Effects of CBR Ligands on MGO-Induced Peritoneal Fibrosis

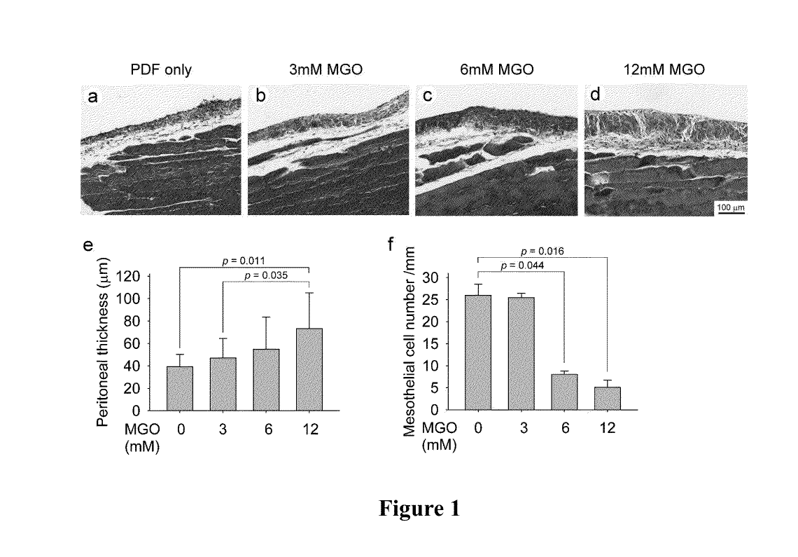

After 10 days of intra-peritoneal injection of PDF with MGO, we found that the rat peritoneum became whitish and less shiny upon gross examination. Peritoneal fibrosis aggravated in rats treated with MGO in a dose-dependent manner, as indicated by increased serosal fibrosis and mesothelium detachment of the peritoneum (FIG. 1). The treatment of the CB₁R antagonist and the CB₂R agonist significantly alleviated peritoneal fibrosis (FIG. 2). Furthermore, we found that MGO-induced mesothelial cell detachment, angiogenesis, and up-regulated TGF-β1, VEGF, and Snail were attenuated by the CB₁R antagonist treatment (FIG. 3).

2. The Effects of CBR Ligands on Mesothelial Cells

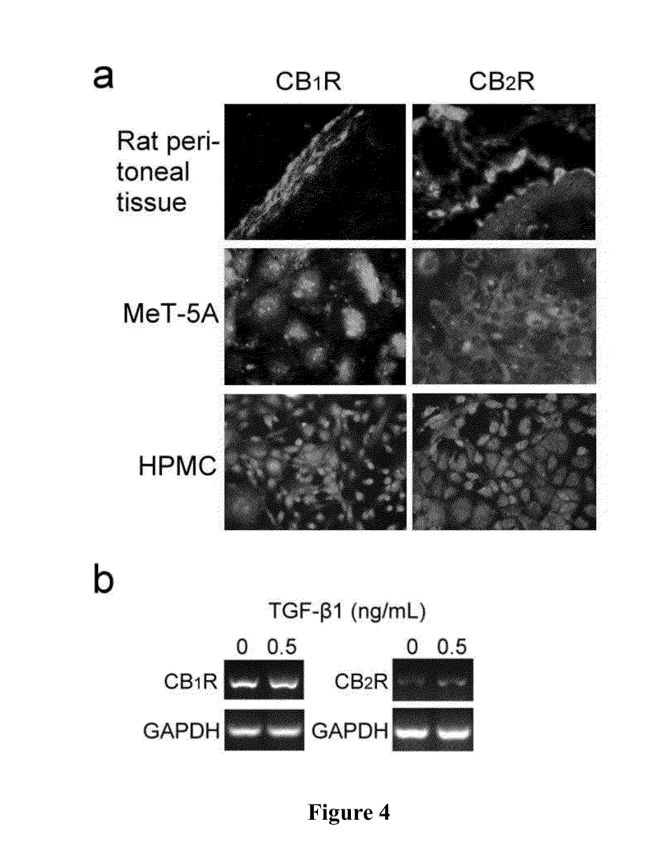

Both CB₁R and CB₂R were expressed on cultured mesothelial cells and on rat peritoneal mesothelium (FIG. 4a). RT-PCR analyses showed that exogenous TGF-β1 had no significant effect on CBR levels in cultured mesothelial cell line (MeT-5A) (FIG. 4b).

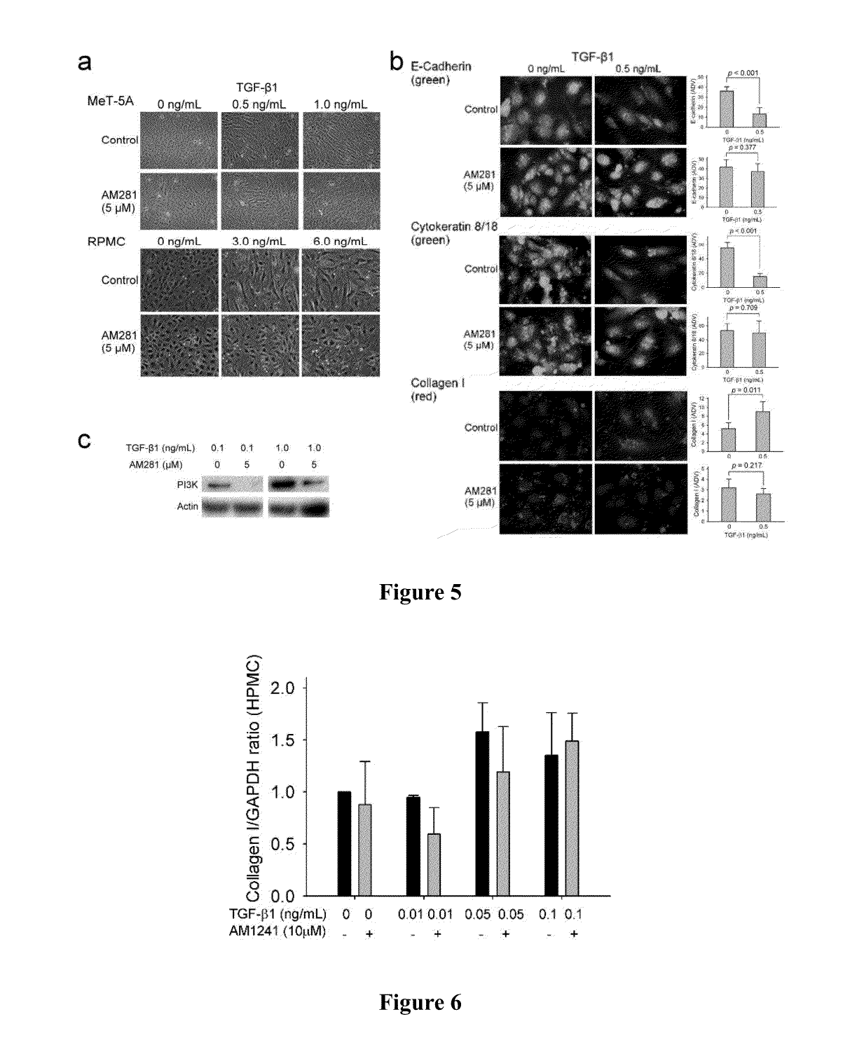

In the presence of TGF-β1, AM281 treatment not only maintained the epithelial integrity by preserving both the epithelial morphology and the expression of cytokeratin 8/18 and E-cadherin, but also inhibited type I collagen synthesis in cultured mesothelial cells (MeT-5A) (FIG. 5a-b). Furthermore, AM281 suppressed TGF-β1-activated PI3K (FIG. 5c).

Although AM1241 tended to suppress type I collagen synthesis of cultured HPMCs under low TGF-β1 concentrations (0.01-0.05 ng/mL), the differences were not statistically significant (FIG. 6). Similar results were found when MeT-5A cells were treated with AM1241 (data not shown).

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

### REFERENCES

1. Gotloib L, Shostak A, Wajsbrot V, Kushnier R: High glucose induces a hypertrophic, senescent mesothelial cell phenotype after long in vivo exposure. Nephron 1999; 82:164-173.
2. Yang A H, Chen J Y, Lin Y P, Huang T P, Wu C W: Peritoneal dialysis solution induces apoptosis of mesothelial cells. Kidney Int 1997; 51:1280-1288.
3. Jin H M, Di Y D, Xu Q J: Effects of commercial glucose-based peritoneal dialysates on peripheral blood phagocytes apoptosis. Perit Dial Int 1999; 19 Suppl 2:S388-S393.
4. Kjellstrand P, Martinson E, Wieslander A, Holmquist B: Development of toxic degradation products during heat sterilization of glucose-containing fluids for peritoneal dialysis: influence of time and temperature. Perit Dial Int 1995; 15:26-32.
5. Nilsson-Thorell C B, Muscalu N, Andren A H, Kjellstrand P T, Wieslander A P: Heat sterilization of fluids for peritoneal dialysis gives rise to aldehydes. Perit Dial Int 1993; 13:208-213.
6. Jones A: Glucose degradation products in peritoneal dialysis: from bench to bedside. Kidney Blood Press Res 2003; 26:113-117.
7. Linden T, Forsback G, Deppisch R, Henle T, Wieslander A: 3-Deoxyglucosone, a promoter of advanced glycation end products in fluids for peritoneal dialysis. Perit Dial Int 1998; 18:290-293.
8. Nakayama M, Sakai A, Numata M, Hosoya T: Hyper-vascular change and formation of advanced glycation endproducts in the peritoneum caused by methylglyoxal and the effect of an anti-oxidant, sodium sulfite. Am J Nephrol 2003; 23:390-394.
9. Riboulet-Chavey A, Pierron A, Durand I, Murdaca J, Giudicelli J, Van O E: Methylglyoxal impairs the insulin signaling pathways independently of the formation of intracellular reactive oxygen species. Diabetes 2006; 55:1289-1299.
10. Hirahara I, Ishibashi Y, Kaname S, Kusano E, Fujita T: Methylglyoxal induces peritoneal thickening by mesenchymal-like mesothelial cells in rats. Nephrol Dial Transplant 2009; 24:437-447.
11. Schalkwijk C G, ter Wee P M, Teerlink T: Reduced 1,2-dicarbonyl compounds in bicarbonate/lactate-buffered peritoneal dialysis (PD) fluids and PD fluids based on glucose polymers or amino acids. Perit Dial Int 2000; 20:796-798.
12. De Vriese A S, Tilton R G, Mortier S, Lameire N H: Myofibroblast transdifferentiation of mesothelial cells is mediated by RAGE and contributes to peritoneal fibrosis in uraemia. Nephrol Dial Transplant 2006; 21:2549-2555.
13. Jimenez-Heffernan J A, Aguilera A, Aroeira L S, Lara-Pezzi E, Bajo M A, del PG, Ramirez M, Gamallo C, Sanchez-Tomero J A, Alvarez V, Lopez-Cabrera M, Selgas R Immunohistochemical characterization of fibroblast subpopulations in normal peritoneal tissue and in peritoneal dialysis-induced fibrosis. Virchows Arch 2004; 444:247-256.
14. Radisky D C: Epithelial-mesenchymal transition. J Cell Sci 2005; 118:4325-4326.
15. Yanez-Mo M, Lara-Pezzi E, Selgas R, Ramirez-Huesca M, Dominguez-Jimenez C, Jimenez-Heffernan J A, Aguilera A, Sanchez-Tomero J A, Bajo M A, Alvarez V, Castro M A, del P G, Cirujeda A, Gamallo C, Sanchez-Madrid F, Lopez-Cabrera M: Peritoneal dialysis and epithelial-to-mesenchymal transition of mesothelial cells. N Engl J Med 2003; 348:403-413.
16. Selgas R, Bajo A, Jimenez-Heffernan J A, Sanchez-Tomero J A, Del P G, Aguilera A, Lopez-Cabrera M: Epithelial-to-mesenchymal transition of the mesothelial cell—its role in the response of the peritoneum to dialysis. Nephrol Dial Transplant 2006; 21 Suppl 2:ii2-ii7.
17. Aroeira L S, Aguilera A, Sanchez-Tomero J A, Bajo M A, del P G, Jimenez-Heffernan J A, Selgas R, Lopez-Cabrera M: Epithelial to mesenchymal transition and peritoneal membrane failure in peritoneal dialysis patients: pathologic significance and potential therapeutic interventions. J Am Soc Nephrol 2007; 18:2004-2013.
18. Stavenuiter A W, Schilte M N, Ter Wee P M, Beelen R H: Angiogenesis in peritoneal dialysis. Kidney Blood Press Res 2011; 34:245-252.
19. Schilte M N, Celie J W, Wee P M, Beelen R H, van den B J: Factors contributing to peritoneal tissue remodeling in peritoneal dialysis. Perit Dial Int 2009; 29:605-617.
20. Demuth D G, Molleman A: Cannabinoid signalling. Life Sci 2006; 78:549-563.
21. Pertwee R G: Cannabinoid pharmacology: the first 66 years. Br J Pharmacol 2006; 147 Suppl 1:S163-S171.
22. Hirahara I, Kusano E, Yanagiba S, Miyata Y, Ando Y, Muto S, Asano Y: Peritoneal injury by methylglyoxal in peritoneal dialysis. Perit Dial Int 2006; 26:380-392.
23. Patel P, Sekiguchi Y, Oh K H, Patterson S E, Kolb M R, Margetts P J: Smad3-dependent and -independent pathways are involved in peritoneal membrane injury. Kidney Int 2010; 77:319-328.
24. Ke Y, Reddel R R, Gerwin B I, Reddel H K, Somers A N, McMenamin M G, LaVeck M A, Stahel R A, Lechner J F, Harris C C: Establishment of a human in vitro mesothelial cell model system for investigating mechanisms of asbestos-induced mesothelioma. Am J Pathol 1989; 134:979-991.
25. Yang A H, Chen J Y, Lin J K: Myofibroblastic conversion of mesothelial cells. Kidney Int 2003; 63:1530-1539.

I/we claim:

1. A method for treating, preventing or reducing peritoneal fibrosis comprising administering to a subject in need thereof a pharmacologically effective dose of a type I cannabinoid receptor (CB₁R) antagonist.

2. The method of claim 1, wherein the CB₁R antagonist is selected from the group consisting of 5-(4-Chlorophenyl)-1-(2,4-dichloro-phenyl)-4-methyl-N-(piperidin-1-yl)-1H-pyrazole-3-carboxamide (SR141716A), 4-[6-methoxy-2-(4-methoxyphenyl)1-benzofuran-3-carbonyl]benzonitrile (LY320135), N-(piperidin-1-yl)-1-(2,4-dichlorophenyl)-5-(4-iodophenyl)-4-methyl-1H-pyrazole-3-carboxamide (AM251), N-(morpholin-1-yl)-1-(2,4-dichlorophenyl)-5-(4-iodophenyl)-4-methyl-1H-pyrazole-3-carboxamide (AM281), Δ9-tetrahydrocannabivarin (THCV), and analogues thereof.

3. The method of claim 2, wherein the CB₁R antagonist is AM281.

4. The method of claim 1, wherein the subject is treated with peritoneal dialysis (PD).

5. A method for treating, preventing or reducing peritoneal fibrosis comprising administering to a subject in need thereof a pharmacologically effective dose of a type II cannabinoid receptor (CB₂R) agonist.

6. The method of claim 5, wherein the CB₂R agonist is selected from the group consisting of (2-iodo-5-nitrophenyl)-[1-[(1-methylpiperidin-2-yl)methyl]indol-3-yl]methanone (AM1241), (6aR,10aR)-3-(1,1-Dimethylbutyl)-6a,7,10,10a-tetrahydro-6,6,9-trimethyl-6H-dibenzo[b,d]pyran (JWH-133), 1-(2,3-Dichlorobenzoyl)-5-methoxy-2-methyl-3-[2-(4-morpholinyl)ethyl]-1H-indole (GW-405,833), [(1R,2R,5R)-2-[2,6-dimethoxy-4-(2-methyloctan-2-yl)phenyl]-7,7-dimethyl-4-bicyclo[3.1.1]hept-3-enyl]methanol (HU-308), Δ9-tetrahydrocannabivarin (THCV), cannabidiol (CBD), and analogues thereof.

7. The method of claim 6, wherein the CB₂R agonist is AM1241.

8. The method of claim 5, wherein the subject is treated with peritoneal dialysis (PD).

9. A method for treating, preventing or reducing peritoneal fibrosis in a subject under peritoneal dialysis (PD) treatment, comprising:
(a) supplementing a peritoneal dialysis fluid (PDF) with a pharmacologically effective dose of a type I cannabinoid receptor (CB₁R) antagonist; and
(b) applying such PDF in said PD treatment.

10. The method of claim 9, wherein the CB₁R antagonist is selected from the group consisting of 5-(4-Chlorophenyl)-1-(2,4-dichloro-phenyl)-4-methyl-N-(piperidin-1-yl)-1H-pyrazole-3-carboxamide (SR141716A), 4-[6-methoxy-2-(4-methoxyphenyl)1-benzofuran-3-carbonyl]benzonitrile (LY320135), N-(piperidin-1-yl)-1-(2,4-dichlorophenyl)-5-(4-iodophenyl)-4-methyl-1H-pyrazole-3-carboxamide (AM251), N-(morpholin-1-yl)-1-(2,4-dichlorophenyl)-5-(4-iodophenyl)-4-methyl-1H-pyrazole-3-carboxamide (AM281), Δ9-tetrahydrocannabivarin (THCV), and an analogue thereof.

11. The method of claim 10, wherein the CB₁R antagonist is AM281.

12. A method for treating, preventing or reducing peritoneal fibrosis in a subject under peritoneal dialysis (PD) treatment, comprising:
(a) supplementing a peritoneal dialysis fluid (PDF) with a pharmacologically effective dose of a type II cannabinoid receptor (CB₂R) agonist; and
(b) applying such PDF in said PD treatment.

13. The method of claim 12, wherein the CB₂R agonist is selected from the group consisting of (2-iodo-5-nitrophenyl)-[1-[(1-methylpiperidin-2-yl)methyl]indol-3-yl]methanone (AM1241), (6aR,10aR)-3-(1,1-Dimethylbutyl)-6a,7,10,10a-tetrahydro-6,6,9-trimethyl-6H-dibenzo[b,d]pyran (JWH-133), 1-(2,3-Dichlorobenzoyl)-5-methoxy-2-methyl-3-[2-(4-morpholinyl)ethyl]-1H-indole (GW-405,833), [(1R,2R,5R)-2-[2,6-dimethoxy-4-(2-methyloctan-2-yl)phenyl]-7,7-dimethyl-4-bicyclo[3.1.1]hept-3-enyl]methanol (HU-308), Δ9-tetrahydrocannabivarin (THCV), cannabidiol (CBD), and an analogue thereof.

14. The method of claim 13, wherein the CB₂R agonist is AM1241.

15. A dialysis fluid for treating, preventing or reducing fibrosis comprising electrolytes, an osmotic agent, physiologically acceptable pH solution, and a pharmacologically effective dose of a type I cannabinoid receptor (CB₁R) antagonist.

16. The dialysis fluid of claim 15, wherein the CB₁R antagonist is selected from the group consisting of 5-(4-Chlorophenyl)-1-(2,4-dichloro-phenyl)-4-methyl-N-(piperidin-1-yl)-1H-pyrazole-3-carboxamide (SR141716A), 4-[6-methoxy-2-(4-methoxyphenyl)1-benzofuran-3-carbonyl]benzonitrile (LY320135), N-(piperidin-1-yl)-1-(2,4-dichlorophenyl)-5-(4-iodophenyl)-4-methyl-1H-pyrazole-3-carboxamide (AM251), N-(morpholin-1-yl)-1-(2,4-dichlorophenyl)-5-(4-iodophenyl)-4-methyl-1H-pyrazole-3-carboxamide (AM281), Δ9-tetrahydrocannabivarin (THCV), and an analogue thereof.

17. The dialysis fluid of claim 16, wherein the CB₁R antagonist is AM281.

18. The dialysis fluid of claim 15, wherein said electrolytes comprise sodium ions, calcium ions, magnesium ions, and chloride ions.

19. The dialysis fluid of claim 18, wherein the electrolytes comprise 130-150 mM of sodium ions, 1-2mM of calcium ions, 0-1 mM of magnesium ions, and 90-110 mM of chloride ions.

20. The dialysis fluid of claim 15, wherein the osmotic agent is one or more compounds selected from the group consisting of monosaccharide, disaccharide, polysaccharide, and amino acid.

21. The dialysis fluid of claim 20, wherein the osmotic agent is glucose or glucose-derived polymer.

22. The dialysis fluid of claim 15, wherein the physiologically acceptable pH solution has a pH of 4.5 to 7.5.

23. A dialysis fluid for treating, preventing or reducing peritoneal fibrosis comprising electrolytes, an osmotic agent, physiologically acceptable pH solution, and a pharmacologically effective dose of a type II cannabinoid receptor (CB₂R) agonist.

24. The dialysis fluid of claim 23, wherein the CB₂R agonist is selected from the group consisting of (2-iodo-5-nitrophenyl)-[1-[(1-methylpiperidin-2-yl)methyl]indol-3-yl]methanone (AM1241), (6aR,10aR)-3-(1,1-Dimethylbutyl)-6a,7,10,10a-tetrahydro-6,6,9-trimethyl-6H-dibenzo[b,d]pyran (JWH-133), 1-(2,3-Dichlorobenzoyl)-5-methoxy-2-methyl-3-[2-(4-morpholinyl)ethyl]-1H-indole (GW-405,833), [(1R,2R,5R)-2-[2,6-dimethoxy-4-(2-methyloctan-2-yl)phenyl]-7,7-dimethyl-4-bicyclo[3.1.1]hept-3-enyl]methanol (HU-308), Δ9-tetrahydrocannabivarin (THCV), cannabidiol (CBD), and analogues thereof.

25. The dialysis fluid of claim 24, wherein the CB₂R agonist is AM1241.

26. The dialysis fluid of claim 23, wherein the electrolytes comprise sodium ions, calcium ions, magnesium ions, and chloride ions.

27. The dialysis fluid of claim 26, wherein the electrolytes comprise 130-150 mM of sodium ions, 1-2 mM of calcium ions, 0-1 mM of magnesium ions, and 90-110 mM of chloride ions.

28. The dialysis fluid of claim 23, wherein the osmotic agent is one or more compounds selected from the group consisting of monosaccharide, disaccharide, polysaccharide, and amino acid.

29. The dialysis fluid of claim 28, wherein the osmotic agent is glucose or glucose-derived polymer.

30. The dialysis fluid of claim 23, wherein the physiologically acceptable pH solution has a pH of 4.5 to 7.5.
